(19) United States
(12) Patent Application Publication
(10) Pub. No.: US 2006/0161065 A1
(43) Pub. Date: Jul. 20, 2006

(54) Similarity scores for electrocardiography

(75) Inventor: Jonathan L. Elion, Wakefield, RI (US)

Correspondence Address: HOUSTON ELISEEVA, 4 MILITIA DRIVE, SUITE 4, LEXINGTON, MA, 02421, US

(73) Assignee: Heartlab, Inc., Westerly, RI (US)

(21) Appl. No.: 11/335,841

(22) Filed: Jan. 18, 2006

Related U.S. Application Data
(60) Provisional application No. 60/644,875, filed on Jan. 18, 2005.

(51) Int. Cl. A61B 5/04

(52) U.S. Cl. 600/509

(57) ABSTRACT

An ECG management system provides a computer-assisted Quality Assurance step in an ECG management system. This step is preferably performed prior to releasing ECGs to the cardiologists for interpretation placement in the patient's permanent records. It involve comparing the ECG wave data to previously collected ECG data for the same and/or different named patients to enable error correction and/or proper patient naming.

### RELATED APPLICATIONS

This application claims the benefit under 35 USC 119(e) of U.S. Provisional Application No. 60/644,875, filed on Jan. 18, 2005, which is incorporated herein by reference in its entirety.

### BACKGROUND OF THE INVENTION

Electrocardiography is a technology for the detection and diagnosis of cardiac conditions. An electrocardiograph is a medical device capable of recording the potential differences generated by the electrical activity of the heart. An electrocardiogram (ECG or EKG) is produced by the electrocardiograph. It typically comprises the ECG wave data that describes the heart's electrical activity as a function of time.

The heart's electrical activity is detected by sensing electrical potentials via a series of electrode leads that are placed on the patient at defined locations on the patient's chest and limbs. Systems with ten (10) separate ECG leads and digital data capture/storage are typical. During electrocardiography, the detected electrical potentials are recorded and graphed as ECG wave data that characterize the depolarization and repolarization of the cardiac muscle.

ECG interpretation is performed by analyzing the various cardiac electrical events presented in the ECG wave data. Generally, the ECG wave data comprise a P wave, which indicates atrial depolarization, a QRS complex, which represents ventricular depolarization, and a T-wave representing ventricular repolarization.

State-of-the-art ECG systems provide for the machine interpretation of the ECG data. These systems are designed to measure features of the ECG wave data from the patient. The various features of portions of the ECG, such as intervals, segments and complexes, including their amplitude, direction, and duration of the waves and their morphological aspects, are measured. Then all of the feature information is analyzed together. From this feature information, these systems are able to generate machine ECG interpretations diagnosing normal and abnormal cardiac rhythms and conduction patterns. These interpretations are often used by the physician/cardiologist as the basis of an ECG report for a given patient.

### SUMMARY OF THE INVENTION

In the typically hospital, ECGs are taken at carts throughout the hospital or institution and received at a records storage location for filing and possibly over-reading by staff cardiologists. In this process, it is important that the ECGs are associated with the proper patient file. Unfortunately, ECGs are often taken in situations where patient names or identifiers have not been provided to the ECG cart, usually from a central computer facility. This means that the nurse or technician, who operates the ECG cart and does the ECG acquisition, has to enter the patient's demographic information (name, medical record number, etc.). This leads to several possibilities for errors, including, but not limited to: 1) the identifiers are incorrect or incomplete; 2) the identifiers are missing; and/or 3) the identifiers from the previous patient to have an ECG are inadvertently used.

When the identifiers are incorrect or incomplete, it is usually possible to reconstruct the correct information using searches of the hospital's patient database. Whereas, when the identifiers are missing or the identifiers from the previous patient are used, traditional information technology is unlikely to help resolve the problem.

When the identifiers are missing, it would be helpful to be able to find another ECG that looks very similar to the one with the missing identifiers. ECGs taken around the same time could be searched. For example, it would be helpful to know that an unlabeled ECG from the emergency room is a close match to an ECG taken on the Coronary Care Unit one hour later. The paper chart can be reviewed, where a paper copy of the ECG may be hand-labeled (but the identifier had not been entered into the computer); this allows complete resolution of the error.

Similarly, when the identifiers from the previous patient are used, it is sometimes apparent that an ECG labeled as belonging to one patient does not match one recorded for that patient before or after the ECG in question. An ability to confirm the difference automatically assists the technicians, who would then consider the ECG in question to not have a valid identifier, and would undertake a corrective procedure.

The present invention functions as part of a comprehensive ECG management System to provide a computer-assisted Quality Assurance step in an ECG management system. It involves comparing ECG data for the same and/or different patients to ensure the accuracy of the ECG patient data. This step is preferably performed prior to releasing ECGs to the cardiologists for interpretation and placement in the patient's permanent records.

In general, according to one aspect, the invention features a method for verifying electrocardiogram (ECG) data in a management system. This method comprises comparing current ECG data to previous ECG data and determining whether similarities or differences between the current ECG data and the previous ECG data suggest error in or correction of patient identification. Then, review is indicated when the similarities or differences suggest such error or possible correction.

In a preferred embodiment, the step of comparing current ECG data to previous ECG data comprises comparing patient demographic data for the current ECG data and the previous ECG data. In other embodiments, the step of comparing current ECG data to previous ECG data further or alternatively comprises assessing differences in the ECG wave data for the current and previous ECG data. In one example, the differences are determined with respect to leading portions of the beats in the ECG wave data. In one example, the step of determining whether the similarities or differences exist comprises determining if the current ECG data are similar to previous ECG data for the same patient. In another example, it is determined if the current ECG data are similar to previous ECG data for a different named patient.

In general, according to another aspect, the invention features a system for verifying electrocardiogram data. The system comprises a patient records database for storing ECG data for patients and a management system for comparing current ECG data to previous ECG data from the patient records database and determining whether similarities or differences exist between the current ECG data and the previous ECG data suggest error in patient identification.

In general, according to another aspect, the invention features a computer software product for ECG data management. The product comprises a computer-readable medium in which program instructions are stored. These instructions, when read by a computer, cause the computer to compare current ECG data to previous ECG data and determine whether similarities or differences exist between the current ECG data and the previous ECG data. When the similarities or differences suggest error in or correction of patient identification, review is indicated.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram illustrating the electrocardiogram (ECG) workflow in a typical hospital;

FIG. 2 is a flow diagram illustrating the machine interpretation process in a conventional ECG device or host-based interpretation system;

FIG. 3 shows prototypical ECG wave data illustrating the various portions of the wave; and

FIG. 4 is a flow diagram illustrating the process for ECG data quality assurance according to the present invention.

### DETAILED DESCRIPTION OF THE PREFERRED EMBODIMENTS

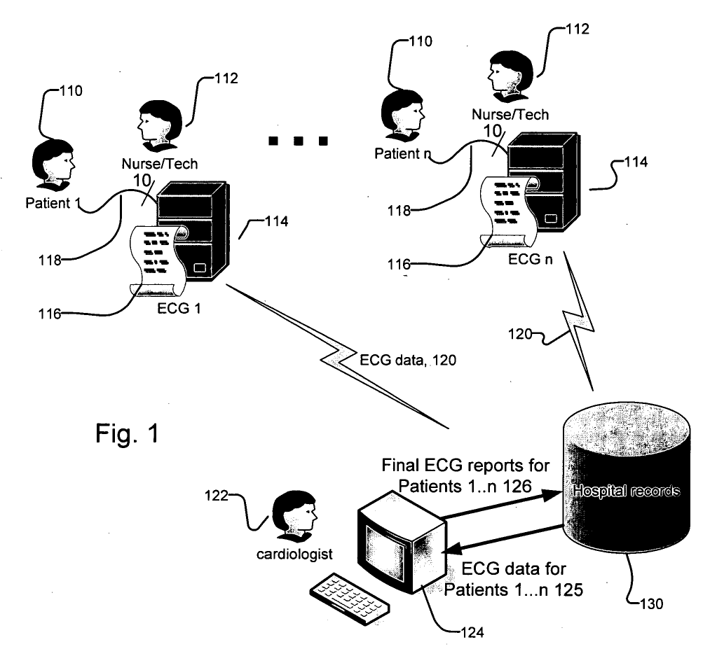

FIG. 1 illustrates the electrocardiogram (ECG) workflow in a typical hospital. A nurse or ECG technician 112-1 interacts with the patient 1 110-1 to acquire the ECG data. In many modem systems, the ECG machine 114-1 is an ECG cart that is moved throughout the hospital between patient, examining, and operating rooms.

In operation, the ten (10) leads 118 of the ECG device 114-1 are placed on the limbs and torso of the patient 110-1. Then, a printout of the ECG wave data 116 is generated at the cart. Also, ECG data 120-1 including the wave data using 12 combinations of the leads that have been placed on the patient and possibly a machine-generated ECG interpretation are generated and digitally stored in the ECG cart 114-1 and/or sent or transmitted to a central hospital records data storage and host system 130.

In parallel, other nurses/technicians 112-n are taking ECGs of other patients 110-n such as patient n. All of the ECG data records 120-n are similarly sent back to the records database and ECG management system 130, which is a central depository database of hospital records and a host system for processing the ECG data from the various patients. Here the ECG data from all of the patients are accumulated.

The present invention generally applies to a comprehensive ECG management system. Such systems will often combine data storage and hostbased interpretation and ECG editing capabilities. In these systems, a cardiologist 122 accesses the ECG data 125 from the records database management system 130 usually via a workstation 124. The hospital records and host system 130 will store preliminary ECG data, generate and store machine interpretations of the ECG data, and store the subsequent final reports 126 that are the product of the editing process by the cardiologist 122 at the workstation 124. The final reports will then be entered into the patients' records.

The workstation 124 is provided with standard software for accessing and editing the ECG data, machine-generated interpretations and reports from host system 130, and generating the final cardiologist-reviewed ECG reports. In the preferred implementation, the database and management system 130 or workstation 124 also has a host-based interpretation system that enables it to generate its own machine-generated interpretation using the ECG data 120 from the cart 114, for example.

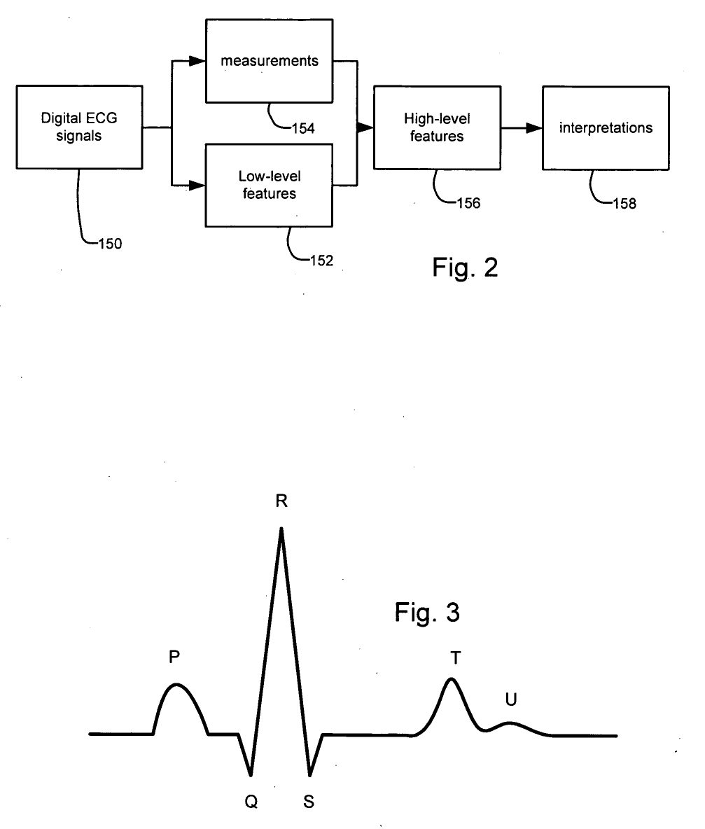

FIG. 2 illustrates the general process by which these machine interpretations are generated. Commonly, they are performed in the cart or in host-based interpretation systems. In either case, the raw ECG wave data are machine interpreted for the cardiologist or other reader.

Specifically, the digital ECG signals or wave data 150 are acquired in step 150 and stored such as by the ECG cart. Measurements of portions of this ECG wave data are made in step 154 and low-level features 152 are typical identified in the wave data at the host system 130. This information is then combined in step 156 where high-level features are determined. Based on these calculated features, the final machine interpretation is generated in step 158.

The features typically relate to the length and amplitude of the various components of a selected ECG wave from one typical cardiac cycle out of the usually very long wave data set that the machine acquires. In other cases, an average ECG wave is calculated from a series of waves to form the basis of the interpretation.

FIG. 3 illustrates a prototypical ECG wave. It generally comprises a P wave, a QRS wave complex, a T-wave, and a U wave. The features that the typical system uses can be dependent on specific characteristics of that system but will include intervals, segments and complexes, including amplitude, direction, and duration of the waves and their morphological aspects.

To some degree, the ECG wave data for an individual are somewhat like a finger print to an experienced cardiologist. Absent a dramatic change in a patient, a cardiologist can determine with some level of certainty whether two ECGs were from the same or different patients. This invention leverages these characteristics of ECGs but in the context of an automated system.

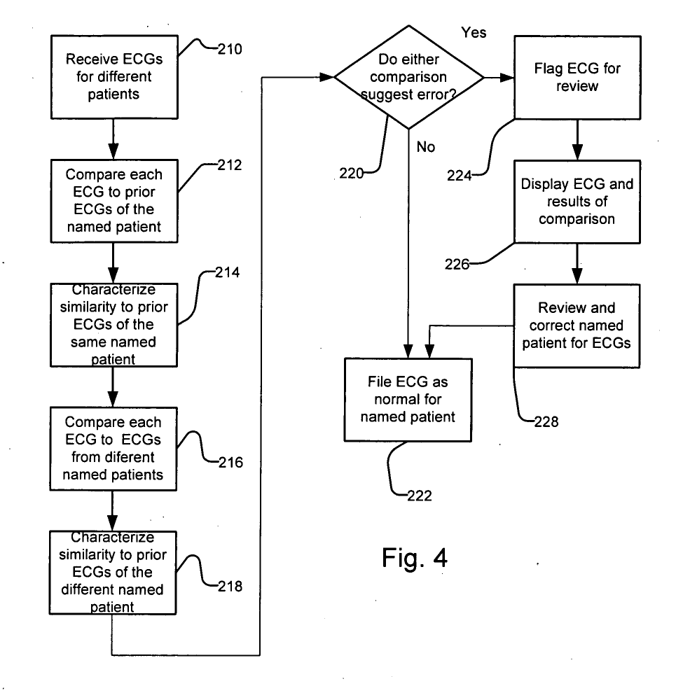

FIG. 4 illustrates a process for ECG quality assurance according to the present invention.

In more detail, the ECG data for different patients are received in step 210 at the management system 130. Typically, this is a central location typically tasked with filing the ECGs and also distributing the ECGs to cardiologist for batch over-reading. The ECGs will typically be generated throughout the hospital, in such varied environments as the emergency room and patient examining rooms.

This ECG data received at the management system 130 include the ECG wave data and patient identification information. The patient identification information is useful for filing the ECG data with the proper patient's file.

According to the invention, the management system 130 performs quality assurance testing. Reasons that an ECG might fail this test include:

1. Signal quality errors found during the initial interpretation step;

2. Demographic information such as age or gender do not match those on the previous ECGs;

3. The patient name does not match the name on the previous ECGs;

4. The ECG is substantially different from the previous ECG for that patient.

This last case suggests the possibility that the ECG might be from the wrong patient (due to failure to reset the patient name in the ECG cart between patients). In order to distinguish an ECG from a totally different patient from the situation where a patient's ECG has legitimately changed from its previous state, a metric is required to determine the degree of similarity between two ECGs.

According to the invention, the database management system 130 compares each of the ECGs to prior ECGs of the named patient. Specifically, when the ECGs are originally taken at the cart, typically the nurse or technician enters the patient name or more typically a patient number or the cart receives the information from a centralized system such as the management system 130. This patient number travels with the ECG data to the database as a mechanism for ensuring that the ECGs are put in the correct patient's file. Specifically, in step 212, a similarity between the new ECGs and prior ECGs for the same named patient is assessed.

Generally, the objective is to bias the comparison to generating false negatives. That is, in step 214, when the system characterizes the similarities, the system should tend to indicate that the named patient on the ECG is or could be wrong even if there is a somewhat strong similarity to previous ECGs from the same patient.

There are several candidate algorithms to assist with computing similarities. Some factors that can be used are based on the actual ECG waveforms (the electrical deflections representing the electrical activity in the heart), and others are based on the interpretation of the waveforms. During the course of a heart attack, for example, the waveform appearance may change considerably from day-to-day, but there are several factors that would remain more constant and therefore more useable for a Similarity Score.

The exemplary algorithms for similarity include:

1. Root Mean Square (RMS) differences between the median beats in each lead of the two ECGs to be compared;

2. Root Mean Square (RMS) differences between the median beats in each lead of the two ECGs to be compared, but restricted to the leading portion of the beats such as the first 40 milliseconds of each beat. This approach looks at the initial electrical vector of each beat, and is most likely to be the same in two ECGs from the same patient, despite ST segment changes that occur later in the beat.

3. Root Mean Square (RMS) differences between the median beats in each lead of the two ECGs to be compared, but with additional weighted factors to increase the similarity score for ECGs taken at close to the same time, or in the same part of the hospital, and with increased uncertainty in the presence of intermittent ventricular pacing or rate-related bundle branch block.

Often, this may be a first ECG for the specific named patient. Thus, there is no prior ECG to generate a comparison. More often, the patient name/number may be invalid or uses a “John Doe” identifier. In still other cases, demographic information in the ECG data may not match data for the named patient. In each of theses situations, there is possibility of or indication of error. As a result, in step 216, the incoming ECGs are also compared to ECGs from different named patients using the exemplary algorithms describe above, for example. Typically, these ECG against which the comparisons are made are ECGs that have been received recently at the database/management system 130.

The relevance of this comparison to ECGs of potentially different patients concerns the fact that it is common, especially in the emergency room environment, that the ECG machines will be moved quickly between patients. Especially in an emergency situation, it may not be that the ECG patient data are updated. In other examples, a “John Doe” name is used where the patient's name is unknown. Comparison of the ECGs to recent ECGs allows for these ECGs to be potentially categorized with the correct named patient or the same “John Doe” patient. Generally, this test is structured to generated false positives, when the characterization of the similarities is made in step 218.

In step 220, a determination is made whether each of the comparisons in steps 212 or 216 suggest error.

In step 224, the ECG is flagged for review if either of the comparisons suggests possible error. In step 226, the results of the flagged ECG comparison is presented to a technician or cardiologist. There, the technician or cardiologist will confirm whether there is in fact similarity. It there is a suggestion that the ECG has an incorrectly named patient or a previous ECG has an incorrectly named patient, then a review is begun in step 228, which can include contacting the individuals responsible for collecting the ECGs to resolve the apparent discrepancy.

Finally, if the comparison suggests no error in step 220, or after research as to whether or not the ECG is correct, the ECG is filed as normally in step 222, either for the named patient or the corrected patient name.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for verifying electrocardiogram (EGG) data in a management system, the method comprising:
comparing current ECG data to previous ECG data;
determining whether similarities or differences between the current ECG data and the previous ECG data suggest error or correction in patient identification; and
indicating review when the similarities or differences suggest error or correction.

2. A method as claimed in claim 1, wherein the step of comparing current ECG data to previous ECG data comprises comparing patient demographic data for the current ECG data and previous ECG data.

3. A method as claimed in claim 1, wherein the step of comparing current ECG data to previous ECG data comprises assessing differences in the ECG wave data for the current ECG data to previous ECG data.

4. A method as claimed in claim 3, wherein the differences are determined with respect to leading portions of beats in the ECG wave data for the current ECG data to previous ECG data.

5. A method as claimed in claim 1, wherein the step of determining whether similarities or differences exist comprises determining if the current ECG data are similar to previous ECG data for the same patient.

6. A method as claimed in claim 1, wherein the step of determining whether similarities or differences exist comprises determining if the current ECG data are similar to previous ECG data for a different named patient.

7. A system for verifying electrocardiogram (ECG) data, the system comprising:
a patient records database for storing ECG data for patients; and
a management system for comparing current ECG data to previous ECG data from the patient records database and determining whether similarities or differences between current ECG data and previous ECG data suggest error in patient identification.

8. A system as claimed in claim 7, wherein the management system compares patient demographic data for the current ECG data and previous ECG data.

9. A system as claimed in claim 7, wherein the management system assesses differences in the ECG wave data for the current ECG data and the previous ECG data.

10. A system as claimed in claim 9, wherein the differences are determined with respect to leading portions of beats in the ECG wave data for the current ECG data and the previous ECG data.

11. A system as claimed in claim 7, wherein the management system determines if the current ECG data are similar to previous ECG data for the same patient.

12. A system as claimed in claim 7, wherein the management system determines if the current ECG data are similar to previous ECG data for a different named patient.

13. A computer software product for ECG data management, the product comprising a computer-readable medium in which program instructions are stored, which instructions, when read by a computer, cause the computer to compare current ECG data to previous ECG data, determine whether a similarities or differences between current ECG data to previous ECG data suggest error in patient identification, and indicate review when the similarities or differences suggest error.

14. A product as claimed in claim 13, wherein the instructions cause the computer to compare patient demographic data for the current ECG data and the previous ECG data.

15. A product as claimed in claim 13, wherein the instructions cause the computer to assess differences in the ECG wave data for the current ECG data and the previous ECG data.

16. A product as claimed in claim 15, wherein the differences are determined with respect to leading portions of beats in the ECG wave data for the current ECG data and the previous ECG data.

17. A product as claimed in claim 13, wherein the instructions cause the computer to determine if the current ECG data are similar to previous ECG data for the same patient.

18. A product as claimed in claim 13, wherein the instructions cause the computer to determine if the current ECG data are similar to previous ECG data for a different named patient.
